(19) United States
(12) Patent Application Publication
(10) Pub. No.: US 2009/0264454 A1
(43) Pub. Date: Oct. 22, 2009

(54) COMBINED HYDROCODONE AND ANALGESIC FORMULATION AND METHOD

(76) Inventor: Robert P. Nickell, Torrance, CA (US)

Correspondence Address: STETINA BRUNDA GARRED & BRUCKER, 75 ENTERPRISE, SUITE 250, ALISO VIEJO, CA, 92656, US

(21) Appl. No.: 12/104,374

(22) Filed: Apr. 16, 2008

(51) Int. Cl. A61K 31/485; A61P 29/00; A61P 25/00

(52) U.S. Cl. 514/282

(57) ABSTRACT

The present invention is directed to co-administration of hydrocodone and a second analgesic agent for the treatment of pain. A pharmaceutical composition suitable for the co-administration contains a therapeutically effective amount of hydrocodone or a pharmaceutically acceptable salt thereof, and a therapeutically effective amount of at least one second analgesic agent. A ratio of the hydrocodone or pharmaceutically acceptable salt thereof to the at least one second analgesic agent in the composition is within a range that provides greater pain relief than that obtainable by the administration of the hydrocodone or second analgesic agent alone. Examples of pharmaceutical compositions for co-administration of the agents are those containing hydrocodone and indomethacin (“Indocodone”), hydrocodone and naproxen (“Naprocodone”), hydrocodone and diclofenac (“Diclodone”) and hydrocodone and tramadol (“Tramacodone”).

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field

The invention generally relates to pharmaceutical compositions for the treatment of pain, and methods of treatment with the pharmaceutical compositions.

2. Related Art

Pain-relief compounds and methods for their use have been developed for the treatment of various different painful conditions, such as conditions involving acute and/or chronic pain. Categories of compounds known to be useful for such treatment include steroidal and non-steroidal anti-inflammatory compounds (NSAIDs), opioids, NMDA antagonists, and other analgesic agents. Non-steroidal anti-inflammatory drugs in particular have been found to be useful in the treatment of pain associated with inflammation, such as rheumatoid arthritis, osteoarthritis, headache and migraine pain, post-operative pain, tissue injury, gout, ileus and other painful inflammatory disorders. The non-steroidal anti-inflammatory drugs are widely-used because they are non-narcotic and typically relatively safe, with certain NSAIDs even being available over-the-counter without a prescription. Examples of popular NSAIDs include aspirin, ibuprofen and naproxen.

However, a problem with the use of NSAIDs is that they have been discovered to cause significant adverse drug reactions in the form of severe gastrointestinal irritation in certain circumstances, such as with very high doses or prolonged administration of the NSAIDs. The gastrointestinal irritation can be serious enough to cause gastric injury, including serious ulcers and gastrointestinal bleeding, even resulting in death. Certain precautions can be taken to reduce the chances of gastric injury, such as by advising patients to take NSAIDs only after consuming a meal and/or drinking water, and by limiting the dose of the NSAID and duration over which it is administered. However, the risk of gastric injury continues to limit the use of NSAID compounds to lower doses and shorter durations of administration than what may otherwise be desired to achieve pain relief. Also, some patients and physicians avoid taking and/or prescribing NSAIDs altogether out of concern for the potential gastrointestinal risks. The limitations of NSAIDs are especially concerning for chronic conditions such as rheumatoid arthritis, which require long-term therapy.

Opioid analgesics are another type of pain-relieving drug that provide fast and effective pain relief that is often superior in overall effect in comparison to other pain-relieving compounds. Popular opioid analgesics include hydrocodone, codeine, oxycodone and morphine, with morphine for example being commonly used in hospital environments for the fast relief of pain resulting from bodily injury/trauma, as well as for the treatment of severe pain associated with terminal cancer and other illnesses. However, the use of opioid analgesics to treat pain can result in a number of undesirable side effects, such as respiratory depression, that can be quite severe. One of the most troubling side effects of opioid analgesic administration is the potential for the development of physiological addiction to the opioid compound. As a result of this potential for addiction, the use of opioid analgesics tends to be limited to only very small dosages of the compounds given over very limited durations, or to treatment in cases of extreme pain where the need for pain relief outweighs any addiction concerns.

Accordingly, there remains a need for pharmaceutical compositions and methods capable of providing effective pain relief without causing significant adverse side effects. There is further a need for pharmaceutical compositions and methods that provide improved pain relief with less potential for addiction. Furthermore, there is a need for compositions and methods that allow for the relatively safe administration of increased doses of pain relievers and/or increased administration duration to provide the desired pain treatment.

### BRIEF SUMMARY OF THE INVENTION

The present invention is directed to co-administration of hydrocodone or a pharmaceutically acceptable salt thereof, and at least one second analgesic agent for the treatment of pain. In one version, a pharmaceutical composition suitable for the co-administration contains a therapeutically effective amount of hydrocodone or a pharmaceutically acceptable salt thereof, and a therapeutically effective amount of at least one second analgesic agent. A ratio of the hydrocodone or pharmaceutically acceptable salt thereof to the at least one second analgesic agent in the composition is within a range that provides greater pain relief than that obtainable by the administration of the hydrocodone or pharmaceutically acceptable salt thereof or at least one second analgesic agent alone.

In one embodiment of the invention, a method for treating pain in a patient in need thereof is provided. The method involves administering to the patient a therapeutically effective amount of hydrocodone or a pharmaceutically acceptable salt thereof, and a therapeutically effective amount of at least one second analgesic agent. The ratio of the hydrocodone or pharmaceutically acceptable salt thereof to the at least one second analgesic agent administered to the patient is maintained in a range that provides greater pain relief than that obtainable by administration of the hydrocodone or pharmaceutically acceptable salt thereof or at least one second analgesic agent alone.

In an embodiment of a pharmaceutical composition for co-administration of the hydrocodone or pharmaceutically acceptable salt thereof and at least one second analgesic agent, the pharmaceutical composition contains the hydrocodone or pharmaceutically acceptable salt thereof in an amount of from about 5 mg to about 15 mg, and contains a second analgesic agent that is at least one of (i) indomethacin or a pharmaceutically acceptable salt thereof in an amount of from about 25 mg to about 75 mg, (ii) naproxen or a pharmaceutically acceptable salt thereof in an amount of from about 200 mg to about 250 mg, (iii) diclofenac or a pharmaceutically acceptable salt thereof in an amount of from about 50 mg to about 75 mg, and (iv) tramadol or a pharmaceutically acceptable salt thereof in an amount of from about 25 mg to about 50 mg. A ratio of the hydrocodone or pharmaceutically acceptable salt thereof to the second analgesic agent is within a range that provides greater pain relief than that obtainable by the administration of the hydrocodone or pharmaceutically acceptable salt thereof or second analgesic agent alone.

The present invention is best understood by reference to the following detailed description.

### DETAILED DESCRIPTION OF THE INVENTION

The detailed description set forth below is intended as a description of the presently preferred embodiments of the invention, and is not intended to represent the only form in which the present invention may be prepared or utilized. The description sets forth the functions and sequences of steps for preparing and using the invention. It is to be understood, however, that the same or equivalent functions may be accomplished by different embodiments and that they are also intended to be encompassed within the scope of the invention.

The expression “pharmaceutically acceptable salt” as used herein is meant to refer to those salts of biological compounds which retain the biological effectiveness and properties of the free compound (i.e. free bases and/or acids), and can include pharmaceutically acceptable acid and/or base addition salts, as well as pharmaceutically acceptable cationic and/or anionic salts. Examples of pharmaceutically acceptable salts include, for example, acid addition salts, such as hydrochloride salts, alkali metal salts, such as sodium and potassium, alkaline earth salts, ammonium salts, and the like.

It should also be understood that the compounds and/or pharmaceutically acceptable salts thereof as described herein may be provided in their hydrate and/or solvate forms.

The expression “therapeutically effective amount” as used herein is meant to refer to an amount of a compound or composition effective to result in the amelioration of symptoms associated with a condition, or to provide a beneficial therapeutic effect, such for example at least partial pain relief, reduction of inflammation, and/or reduction in gastrointestinal irritation.

The expression “gastrointestinal irritation” as used herein is meant to refer to at least one of dyspeptic symptoms, gastroduodenal ulcers, peptic ulcers, perforation of ulcers, gastropathy, upper and/or lower gastrointestinal hemorrhaging, gastroduodenal damage, ulcer complications, stomach erosions and the like.

The expression “co-administration” as used herein is meant to refer to the administration of at least two compounds within the same time frame, such as substantially simultaneously. The expression can refer to the administration of at least two compounds in the same dosage form, substantially simultaneous administration in separate dosage forms, or sequential administration of the compounds within a timeframe selected such that the therapeutic effects of the compounds temporally overlap.

The term “patient” as used herein is meant to refer to a human or non-human mammal capable of receiving treatment with the compositions and methods taught herein.

The term “synergistic effect” as used herein is meant to refer to a therapeutic or other effect achieved by the co-administration of two or more compounds that exceeds a mere additive effect of the compounds.

It has been surprisingly discovered that improved pain relief can be provided by combining the particular opioid that is hydrocodone, or a pharmaceutically acceptable salt thereof, with at least one second analgesic agent. The combination of hydrocodone with the second analgesic agent unexpectedly provides synergistic results in the treatment of pain, as the reduction in pain levels achieved with the combination exceeds a mere additive contribution from each of the compounds. In particular, it has been discovered that the hydrocodone and at least one second analgesic agent can be co-administered in a ratio that is within a range that provides greater pain relief than that obtainable by the administration of either of the hydrocodone or second analgesic agent alone. In addition, adverse side effects normally associated with the administration of hydrocodone are decreased by administering the hydrocodone and second analgesic agent together, resulting in improved treatment of pain with reduced likelihood of addiction and other undesirable side effects.

Hydrocodone is an opioid analgesic agent that provides pain relieve via agonism of opioid receptors. Hydrocodone can be provided as the compound corresponding to the chemical formula 4,5α-Epoxy-3-methoxy-17-methylmorphinan-6-one tartrate (1:1) hydrate (2:5), as well as in other salt and/or hydrate forms. Hydrocodone and pharmaceutically acceptable salts thereof are further described in U.S. Pat. No. 2,715,626, which is herein incorporated by reference in its entirety. It should be understood that the term “hydrocodone” as used herein is intended to encompass not only the free compound (non-salt form), but also pharmaceutically acceptable salts thereof.

The second analgesic agent used for co-administration with the hydrocodone can be selected in relation to the particular condition being treated, and preferably has proven efficacy in the treatment of pain without significant potential for addiction. In one version, the second analgesic agent comprises a non-steroidal anti-inflammatory agent (NSAID), which is a compound that is capable of relieving pain, and especially pain associated with inflammation. Examples of NSAIDs (and their brand-names) suitable for co-administration with the hydrocodone include at least one of diclofenac (Cataflam®, Voltaren®, Voltaren SR®), etodolac (Lodine®, Lodine XL®), ibuprofen (Motrin®), fenoprofen (Nalfon®), indomethacin (Indocin®), ketoprofen (Orudis®, Oruvail®), nabumetome (Relafan®), naproxen (Naprosyn®), oxaprozin (Daypro®), sulindac (Clinoril®) and tolmetin (Tolectin®), as well as pharmaceutically acceptable salts of these compounds. For example, the NSAID co-administered with the hydrocodone can comprise at least one of indomethacin, naproxen, diclofenac and pharmaceutically acceptable salts thereof. The co-administration of a second analgesic agent that is an NSAID with the hydrocodone has the added benefit of reducing symptoms of gastrointestinal irritation that might otherwise occur with administration of the NSAID alone.

In yet another version, the second analgesic agent comprises a compound having at least partial opioid agonist activity, but which also exhibits substantially reduced potential for addiction as compared to typical opioid compounds. For example, a suitable compound having at least partial opioid agonist activity is the compound known as tramadol, corresponding to (1RS,2RS) trans-2-[(dimethylamino)-methyl]-1-(3-methoxyphenyl) cyclohexanol, or a pharmaceutically acceptable salt thereof, as described for example in U.S. Pat. No. 3,652,589, which is herein incorporated by reference in its entirety. Although the mode of action of tramadol is not yet fully understood, the compound is generally understood to be an “atypical” opioid in that it is believed to provide pain relief via mild agonism of the μ-opioid receptor, as well as through modulation of the GABAergic, noradrenergic and serotonergic systems, and may also having some NMDA-type antagonistic effects. Co-administration of tramadol with hydrocodone provides pain relief with substantially and unexpectedly reduced narcotic/addictive effects, and also results in a substantial reduction in side effects commonly associated with the administration of opioid agonists, such as a reduction in the incidence of respiratory depression.

The relative amounts of the hydrocodone and second analgesic agent administered to the patient are selected according to criteria such as the condition to be treated, the particular second analgesic agent being administered, the extent to which the potential for addiction is a concern, the chronic or acute nature of the condition, and other similar criteria. In general, the relative amounts of the compounds are selected to provide for synergistic pain relief. For example, a suitable ratio of hydrocodone to indomethacin may be in the range of from about 1 part by weight of the hydrocodone to from about 1.6 to about 15 parts by weight of the indomethacin. A suitable ratio of hydrocodone to naproxen may be in the range of from about 1 part by weight of the hydrocodone to from about 13 to about 50 parts by weight of the naproxen. As another example, a suitable ratio of hydrocodone to diclofenac may be in the range of from about 1 part by weight of the hydrocodone to from about 3.3 to about 15 parts by weight of the diclofenac. As a final example, a suitable ratio of hydrocodone to tramadol may be in the range of from about 1 part by weight of the hydrocodone to from about 1.6 to about 10 parts by weight of the tramadol. While these ratios are calculated with respect to the free compounds (non-salt forms), it should be understood that the equivalent ratios can also readily be determined for pharmaceutically acceptable salts of the compounds by using a ratio of the molecular weights of the salts, as known by those of ordinary skill in the art. These ranges have been discovered to provide greater pain relief, with less potential for addiction, than what would otherwise be obtainable by the administration of either the hydrocodone or second analgesic agent alone. In other words, the administration of these compounds exhibits synergistic effects that exceed the mere additive contribution of the individual components.

Suitable dosages of the hydrocodone and second analgesic agent for co-administration are similarly selected according to the painful condition to be treated, as well as to provide for the synergistic effects in terms of pain relief and reduced likelihood of addiction. Generally, a suitable dosage of the hydrocodone may range from about 5 mg to about 15 mg, whereas a second analgesic agent for co-administration with the hydrocodone is selected according to the particular second analgesic agent and/or treatment regimen being provided. For example, a suitable treatment regimen can comprise co-administering hydrocodone or a pharmaceutically acceptable salt thereof in a dosage of from 5 mg to about 15 mg, with indomethacin or a pharmaceutically acceptable salt thereof in a dosage of from about 25 mg to about 75 mg. In another version, a suitable treatment regimen can comprise co-administering hydrocodone or a pharmaceutically acceptable salt thereof in a dosage of from 5 mg to about 15 mg, with naproxen or a pharmaceutically acceptable salt thereof in a dosage of from about 200 mg to about 250 mg. In yet version, a treatment regimen comprises co-administering hydrocodone or a pharmaceutically acceptable salt thereof in a dosage of from 5 mg to about 15 mg, with diclofenac or a pharmaceutically acceptable salt thereof in a dosage of from about 50 mg to about 75 mg. In still another version, the treatment regimen comprises co-administering hydrocodone or a pharmaceutically acceptable salt thereof in a dosage of from 5 mg to about 15 mg, with tramadol or a pharmaceutically acceptable salt thereof in a dosage of from about 25 mg to about 50 mg.

The hydrocodone and second analgesic agent are co-administered to treat patients suffering from any of a variety of different painful, including acute as well as chronic conditions. For example, the compounds can be co-administered to treat painful conditions including, but not limited to musculo-skeletal injury, soft tissue injury, dental pain, post-operative pain, port partum pain, surgical pain, dysmenorrheal, migraine, tension headache, sinus headache and neuralgia. The compounds can also optionally be co-administered to treat pain associated with inflammation in arthritic conditions, such as at least one of rheumatoid arthritis, Still's disease, osteoarthritis, other arthritic conditions. Patients treatable by co-administration of the compounds include human patients suffering from these and other painful conditions. Veterinary patients suffering from painful conditions, such as for example any of dogs, cats, horses, livestock and the like, may also receive the hydrocodone and second analgesic agent co-administration treatment.

In one version, co-administration of the hydrocodone and second analgesic agent is achieved by formulating the compounds into a pharmaceutical composition. The pharmaceutical composition comprises a dosage form suitable for any of a number of different means of administration, including but not limited to oral, buccal, parenteral, topical, transdermal, rectal, intravenous, intraperitoneal and inhalable dosage forms. For example, the dosage forms can comprise solid dosage forms, such as at least one of powders, granules, tablets, capsules (e.g. hard and soft gelatin capsules), caplets, cachets, suppositories and pessaries. The dosage forms can also be provided in liquid form, such as for example as solutions, suspensions, emulsions, syrups, elixirs and even pressurized compositions. Other dosage forms can include transdermal forms, such as transdermal patches, as well as aerosolizable forms suitable for pulmonary administration. Sustained release dosage forms can also be provided. The dosage forms typically comprise dosage units, such as tablets or caplets, which contain the appropriate dosage of the hydrocodone and second analgesic agent for administration to the patient. Each unit dosage form can comprise up to about 99% by weight of the combined hydrocodone and second analgesic agent, such as from about 0.03% to about 99% by weight, and even from about 1 to about 80% by weight.

Examples of solid dosage forms of the pharmaceutical composition are those including a pharmaceutically acceptable carrier, and also optionally including other substances such as at least one of a flavoring agent, filler, compression aid, binders, disintegrants and encapsulating materials. Suitable carriers and/or other ingredients suitable for solid dosage forms can include, for example, at least one of calcium phosphate, magnesium stearate, talc, sugars, hydrous lactose, anhydrous lactose, ribose, dextrin, starch, gelatin, cellulose, methyl cellulose, carboxymethyl cellulose, microcrystalline cellulose, starch glycolate, polyvinylpyrrolidine, polymers of methacrylic acid and divinylbenzene, waxes and ion exchange resins, among others. In the formulation of powder solid dosage forms, the carrier, active ingredients and optional auxiliary ingredients are finely divided and mixed together, and used to fill capsules, sachets, and the like. In the formulation of tablet solid dosage forms, the active ingredients are mixed with a carrier having suitable compression properties, and then compressed into a desired tablet shape and size. Spray-drying techniques can also be used to provide granules suitable for incorporation into capsules or compression into tablets.

Examples of liquid forms of the pharmaceutical composition are those comprising liquid carriers, such as for example water, organic solvents, pharmaceutically acceptable oils and/or fats, and combinations thereof, in which one or more of the active agents are dissolved or suspended. The liquid forms optionally further comprise other suitable pharmaceutically acceptable additives such as solubilizers, emulsifiers, buffering agents, preservatives, sweeteners, flavoring agents, suspending agents, thickening agents, coloring agents, viscosity regulators, stabilizers, osmoregulators, and the like. Some examples of liquid forms suitable for oral administration include liquid compositions having water as a carrier and including additives such as cellulose derivatives including carboxymethyl cellulose solutions, compositions having an alcoholic carrier and including mono and polyhydridic alcohols such as glycerin and non-toxic glycols, and also liquid forms comprising pharmaceutically acceptable oils as a carrier, such as coconut oil, safflower oil and/or arachis oil.

The dosage form can be provided in a regimen as prescribed by a physician or veterinarian depending upon the needs of the patient. As an example, a suitable regimen may comprise the administration of one dosage unit (e.g. a tablet and/or capsule) two to four times per day according to the severity of the pain and the responsiveness of the patient to the medication.

While formulation of the hydrocodone and second analgesic agent has been described with regards to the combination of the compounds into a single formulation, it should also be understood that the compounds could be co-administered in separate preparations, such as a first unit dosage form comprising the hydrocodone, and a second unit dosage form suitable for co-administration with the first unit dosage form comprising the second analgesic agent. Other methods or modes of co-administration not specifically described herein should also be understood to be encompassed by the instant invention.

### EXAMPLES

Preferred embodiments of pharmaceutical compositions suitable for co-administration of the hydrocodone with a second analgesic agent are described in more detail in the following examples. It should be understood that these examples are meant for illustrative purposes only, and are in no way intended to limit the scope of the invention thereto.

### Example 1

In this example, a method of preparing capsule formulations for the co-administration of tramadol hydrochloride and hydrocodone bitartrate is described. A batch of the formulation is prepared by providing 3.75 grams of tramadol hydrochloride powder, 1 gram of hydrocodone bitartrate, USP powder, 9.6667 grams of lactose monohydrate spray dried powder and 0.05 grams of riboflavin (vitamin B2), USP powder. The ingredients are combined in a mortar using the principles of geometric dilution, and triturated well to reduce particle size. Once the ingredients have been combined and reduced to the desired particle size, the mixture is poured evenly into 100 separate capsules, resulting in capsule unit dosage forms each having 37.5 mg of tramadol hydrochloride and 10 mg of hydrocodone bitartrate (“tramacodone”).

### Example 2

In this example, a method of preparing another capsule formulation for the co-administration of tramadol hydrochloride and hydrocodone bitartrate is described. A batch of the formulation is prepared by providing 3.75 grams of tramadol hydrochloride powder, 0.5 grams of hydrocodone bitartrate, USP powder, 10.1667 grams of lactose monohydrate spray dried powder and 0.05 grams of riboflavin (vitamin B2), USP powder. The ingredients are combined in a mortar using the principles of geometric dilution, and triturated well to reduce particle size. Once the ingredients have been combined and reduced to the desired particle size, the mixture is poured evenly into 100 separate capsules, resulting in capsule unit dosage forms each having 37.5 mg of tramadol hydrochloride and 5 mg of hydrocodone bitartrate (“Tramacodone).

Additional modifications and improvements of the present invention may also be apparent to those of ordinary skill in the art. Thus, the particular combination of compounds and methods of administration described and illustrated herein is intended to represent only certain embodiments of the present invention, and is not intended to serve as limitations of alternative devices and methods within the spirit and scope of the invention. Along these lines, it should be understood that other combinations of second analgesic agents other than those specifically described can also be used. Also, a pharmaceutical composition used for co-administration of the agents may take any of a variety of dosage forms that are known or later developed in the art. Also, it should be understood that different dosages and/or ratios of the hydrocodone and second analgesic agent other than those specified may be used depending on the nature and synergistic potential with hydrocodone of each particular analgesic agent being used.

What is claimed is:

1. A pharmaceutical composition for the treatment of pain, the composition comprising:
(a) a therapeutically effective amount of hydrocodone or a pharmaceutically acceptable salt thereof; and
(b) a therapeutically effective amount of at least one second analgesic agent,
wherein a ratio of the hydrocodone or pharmaceutically acceptable salt thereof to the at least one second analgesic agent is within a range that provides greater pain relief than that obtainable by the administration of the hydrocodone or pharmaceutically acceptable salt thereof or second analgesic agent alone.

2. The pharmaceutical composition of claim 1 wherein the second analgesic agent comprises a non-steroidal anti-inflammatory agent.

3. The pharmaceutical composition of claim 2 wherein the second analgesic agent comprises at least one of indomethacin, naproxen, diclofenac and pharmaceutically acceptable salts thereof.

4. The pharmaceutical composition of claim 3 comprising indomethacin or a pharmaceutically acceptable salt thereof in an amount of from about 25 mg to about 75 mg, and hydrocodone or a pharmaceutically acceptable salt thereof in an amount of from about 5 mg to about 15 mg.

5. The pharmaceutical composition of claim 3 comprising naproxen or a pharmaceutically acceptable salt thereof in an amount of from about 200 mg to about 250 mg, and hydrocodone or a pharmaceutically acceptable salt thereof in an amount of from about 5 mg to about 15 mg.

6. The pharmaceutical composition of claim 3 comprising diclofenac or a pharmaceutically acceptable salt thereof in an amount of from about 50 mg to about 75 mg, and hydrocodone or a pharmaceutically acceptable salt thereof in an amount of from about 5 mg to about 15 mg.

7. The pharmaceutical composition of claim 1 wherein the second analgesic agent has at least partial activity as an opioid receptor agonist.

8. The pharmaceutical composition of claim 7 wherein the analgesic agent comprises tramadol or a pharmaceutically acceptable salt thereof.

9. The pharmaceutical composition of claim 8 comprising tramadol or a pharmaceutically acceptable salt thereof in an amount of from about 25 mg to about 50 mg, and hydrocodone or a pharmaceutically acceptable salt thereof in an amount of from about 5 mg to about 15 mg.

10. The pharmaceutical composition of claim 1, wherein the composition is in a dosage form that comprises at least one of oral, buccal, parenteral, topical, transdermal, rectal, intravenous, intraperitoneal and inhalable form.

11. The pharmaceutical composition of claim 11 wherein the dosage form comprises from 0.03% to 99% by weight of the second analgesic agent and hydrocodone or pharmaceutically acceptable salt thereof.

12. The pharmaceutical composition of claim 11 wherein the composition is in a solid dosage form, and comprises a pharmaceutically acceptable carrier comprising at least one of calcium phosphate, magnesium stearate, talc, sugars, hydrous lactose, anhydrous lactose, ribose, dextrin, starch, gelatin, cellulose, methyl cellulose, carboxymethyl cellulose, microcrystalline cellulose, starch glycolate, polyvinylpyrrolidine, polymers of methacrylic acid and divinylbenzene, waxes and ion exchange resins.

13. The pharmaceutical composition of claim 12 wherein the solid dosage form comprises at least one of a powder, granules, tablet, capsule, suppository and pessary.

14. A method for treating pain in a patient in need thereof, the method comprising:
administering to said patient:
(i) a therapeutically effective amount of hydrocodone or a pharmaceutically acceptable salt thereof; and
(ii) a therapeutically effective amount of at least one second analgesic agent,
wherein a ratio of the hydrocodone or pharmaceutically acceptable salt thereof to the at least one second analgesic agent is within a range that provides greater pain relief than that obtainable by the administration of the hydrocodone or pharmaceutically acceptable salt thereof or second analgesic agent alone.

15. The method of claim 14, wherein the patient is suffering from at least one of rheumatoid arthritis, Still's disease, osteoarthritis, other arthritic conditions, pain associated with musculo-skeletal injury, soft tissue injury, dental pain, post-operative pain, port partum pain, surgical pain, dysmenorrheal, migraine, tension headache, sinus headache and neuralgia.

16. The method of claim 14, wherein the second analgesic agent comprises a non-steroidal anti-inflammatory agent.

17. The method of claim 14 wherein the second analgesic agent has at least partial activity as an opioid receptor agonist.

18. The method of claim 14 wherein the second analgesic agent comprises at least one of indomethacin, naproxen, diclofenac, tramadol, and pharmaceutically acceptable salts thereof.

19. The method of claim 14 comprising administering the hydrocodone or pharmaceutically acceptable salt thereof and second analgesic agent by at least one of oral, buccal, parenteral, topical, transdermal, rectal, intravenous, intraperitoneal and inhalable route.

20. A pharmaceutical composition for the treatment of pain and inflammation with reduced gastrointestinal irritation, the composition comprising:
(a) hydrocodone or a pharmaceutically acceptable salt thereof in an amount of from about 5 mg to about 15 mg; and
(b) a second analgesic agent comprising at least one of (i) indomethacin or a pharmaceutically acceptable salt thereof in an amount of from about 25 mg to about 75 mg, (ii) naproxen or a pharmaceutically acceptable salt thereof in an amount of from about 200 mg to about 250 mg, (iii) diclofenac or a pharmaceutically acceptable salt thereof in an amount of from about 50 mg to about 75 mg, and (iv) tramadol or a pharmaceutically acceptable salt thereof in an amount of from about 25 mg to about 50 mg,
wherein a ratio of the hydrocodone or pharmaceutically acceptable salt thereof to the second analgesic agent is within a range that provides greater pain relief than that obtainable by the administration of the hydrocodone or pharmaceutically acceptable salt thereof or second analgesic agent alone.
